(19) United States
(12) Patent Application Publication
(10) Pub. No.: US 2023/0285697 A1
(43) Pub. Date: Sep. 14, 2023

(54) DEVICE FOR DELIVERY OF A COMPOUND TO SPECIFIC REGIONS OF THE NASAL CAVITY

(71) Applicants: Andrew Robert Cooper, Woodbury, MN (US); Christopher George Blatchford, Woodbury, MN (US); Stephen Howgill, Woodbury, MN (US); Barzin Gavtash, Woodbury, MN (US); Benjamin James Myatt, Woodbury, MN (US); Hendrik Versteeg, Woodbury, MN (US); Kerry Ward, Woodbury, MN (US); Lee Michael Hodges, Woodbury, MN (US); Sam Oladele Apoola, Woodbury, MN (US); John Paul Bunting, Woodbury, MN (US); KINDEVA DRUG DELIVERY L.P., Woodbury, MN (US)

(72) Inventors: Andrew Robert Cooper, Leicester (GB); Christopher George Blatchford, Melton Mowbray (GB); Stephen Howgill, Leicester (GB); Barzin Gavtash, Loughborough (GB); Benjamin James Myatt, Leicester (GB); Hendrik Versteeg, Loughborough (GB); Kerry Ward, Fortrose (GB); Lee Michael Hodges, Clitheroe (GB); Sam Oladele Apoola, London (GB); John Paul Bunting, Castle Donington (GB)

(21) Appl. No.: 18/018,365

(22) PCT Filed: Jul. 28, 2021
(86) PCT No.: PCT/US2021/043424

§ 371 Date: Jan. 27, 2023

(30) Foreign Application Priority Data
Jul. 29, 2020 (GB) 2011729.7

(51) Int. Cl. A61M 15/08; A61M 15/00

(52) U.S. Cl. CPC A61M 15/08; A61M 15/0065; A61M 15/0086

(57) ABSTRACT

Various embodiments of a device for delivery of a medicament and a method of utilizing such device are disclosed. The device can include a canister and an actuator. The actuator includes a housing receiving the canister; a valve block receiving a metering valve of the canister, the valve block defining an expansion chamber for passage of propellant and medicament expelled from the canister; and a nosepiece for insertion into a user's nostril, the nosepiece including a fluid passage and a delivery opening for expulsion of the propellant and medicament into the user's nostril. A flow path is defined through the actuator for passage of propellant and medicament from the canister and includes the expansion chamber, the fluid passage and the delivery opening. Further, the cross-sectional area of the flow path is greater than the lowest cross-sectional area of the metering valve at all points along the flow path.

### TECHNICAL FIELD

The present disclosure relates to a device for delivery of a compound to at least one specific region of the nasal cavity. More specifically, the present disclosure relates to a nasal pMDI (pressurised metered dose inhaler) for delivering medicaments (also known as active pharmaceutical ingredients, or APIs) to one of the olfactory region, the turbinates region and the nasopharynx of the nasal cavity.

It will be noted that although the term ‘inhaler’ is used in this application to describe the type of device (e.g., pressurised metered dose inhaler), operation of the device does not necessarily require the act of inhalation.

### BACKGROUND ART

There are many conditions and disorders of the central nervous system (CNS) that require delivery of drugs to the brain. Diseases such as Alzheimer's can benefit significantly from the use of drugs introduced to the tissues of the brain.

Unfortunately, delivery of drugs to the brain is problematic. The brain is a highly sensitive and well protected organ, and techniques for drug delivery can therefore be invasive, requiring a high degree of medical intervention, as well as significant inconvenience and discomfort to the patient. The blood-brain barrier (BBB) tends to impede the transport of molecules to the brain.

These technical challenges have resulted in promising medicaments being discarded due to the difficulty in delivering them to the brain, and consequent under funding of this important area of medicine.

One area where the brain is in contact with the exterior of the body is in the nasal cavity. The olfactory nerve extends from the brain into the nasal cavity where direct contact is made between these nerves and the cavity, penetrating the mucosal lining. Millions of neurons are present at this location, facilitating the subject's sense of smell. It has been suggested that these neurons may offer a route to deliver medicaments from the nasal cavity to the brain (the ‘nose-to-brain’ or ‘N2B’ route).

Importantly, use of this route would bypass the otherwise prohibitive blood-brain barrier. Nose to brain transport is therefore seen as an important potential route to deliver important drugs to the CNS.

Although the olfactory nerve endings offer this potential, they are still not easily accessed. They are positioned well into, and on the superior surface of, the nasal cavity. The region is situated in a region of complex and narrow geometry. Therefore, there are challenges in designing a mechanism to deliver medicaments to this surface.

One such solution that has been proposed is in the adaption and application of nasal inhalers. These devices introduce spray or aerosolised medicaments into the nasal cavity. They are used in several medical applications, most commonly for the topical application of medicaments locally to the outer nasal cavity. For example, nasal inhalers are used to treat conditions such as hay fever by delivering a mild steroid to the nasal tissues, thus reducing their tendency to swell in reaction to allergens. It has been suggested that these inhalers may be used for systematic introduction of medicaments, for example to the CNS via the N2B route.

WO 03/090812 to OptiNose AS describes a nasal delivery device that is proposed for use in delivery of medicaments via the N2B route. WO 03/090812 describes a nasal delivery device comprising a chamber, a nosepiece in communication with the chamber, the nosepiece for insertion into a nostril, and a mouthpiece also in communication with the chamber. A delivery unit is provided for delivering an aerosol spray of a propellant containing a medicament into the chamber.

The nosepiece of WO 03/090812 is inserted into the nostril of the user, and the mouthpiece into the mouth. The user then exhales into the mouthpiece. This closes the user's oropharyngeal valve whilst delivering an air flow into the chamber from the mouth to the nose via the nosepiece. When a predetermined flow rate is achieved in this manner, the delivery unit is activated, releasing the medicament. The medicament is entrained into the airflow in the chamber and is carried from the nosepiece into the nasal cavity. The authors of WO 03/090812 claim that the interacting flow from the user's mouth to the nasal cavity provides a spray with optimised characteristics such as an optimum particle size distribution and much reduced velocity. Reduction in velocity is a key object of the device of WO 03/090812, and the arrangement of the device facilitates this objective. WO 03/090812 clearly states that substantial reduction in velocity of the medicament from the delivery unit to the nostril is desirable. It focusses on optimum particle size distribution. It should also be noted that this device is suitable for treatment of nasal conditions other than using the N2B mechanism, and as such offers a ‘one size fits all’ approach.

Devices of the type described in WO 03/090812 will be referred to as exhalation breath-actuated type nasal inhaler devices. One thing such devices have in common is the reduced velocity of the medicament, resulting from a small spray orifice and entrainment in the exhaled air.

US 2016/0279357 to OptiNose AS also describes a nasal delivery device of the exhalation breath-actuated type. This document discloses the use of an inflatable cuff to provide a fluid-tight seal between the nosepiece and the inner surface of the nostril. The document seeks to reduce the spray or aerosol particle size in order to facilitate entry of those particles through the small passages in the olfactory region of the nasal cavity. The document also states that it is essential to close the palatal velum in order to avoid particles being drawn to the inferior and middle regions of the nasal cavity.

WO 2012/119153 to Impel Neuropharma Inc. discloses a device for delivering a compound to the olfactory region of the nasal cavity. The device comprises a metered dose inhaler having a pressurised propellant container and a metering valve. A diffuser (e.g., in the form of a frit) is provided downstream of the propellant container. Downstream of the diffuser is a drug capsule containing a compound chamber in fluid communication with the propellant container's metering valve and with a nosepiece.

Fundamentally, the inhaler of WO 2012/119153 is configured with the propellant and drug kept in separate locations. The propellant is diffused without the drug (which sits downstream in the compound chamber) and the diffused fluid entrains the drug for ejection into the user's nostril. For the purposes of this application this type of device will be referred to as a separated propellant/compound type device. The manufacturer refers to this device as a “precision olfactory delivery” or “POD” device.

US 2016/0101245 to Impel Neuropharma Inc. discloses a unit dose container for the containment of an intranasal formulation for use with the device described in general in WO 2012/119153.

US 2018/0126101 to Impel Neuropharma Inc. discloses a nozzle for use in delivering a mixture of propellant and drug to the nasal cavity.

As mentioned above, traditional aqueous nasal sprays typically deposit drugs close to the nasal valve, not deep in the nasal cavity where locally acting drugs may be required. As well as delivery to the olfactory region, it can also be desirable to introduce medicaments into the other, deeper, regions of the nasal cavity.

The nasopharynx is the uppermost part of the throat, lying above the oral cavity, extending after the choanae in the nasal cavities. It will be noted that the nasopharynx is described herein as part of the nasal cavity.

Targeted nasopharynx drug delivery has potential for therapies for both cancer and viruses located in this region e.g., the Covid-19 virus (SARS-CoV-2).

It may also be desirable to introduce medicaments to the turbinates region of the nasal cavity as well.

It is an aim of the present disclosure to provide an improved nasal inhaler for the delivery of medicaments to the deeper regions of the nasal cavity including the olfactory region, the turbinates region and the nasopharynx. It is also an aim to provide a device that can facilitate delivery via other routes, such as local and systemic routes, as well as via the blood brain barrier and the trigeminal nerve for N2B.

### SUMMARY OF DISCLOSURE

The inventors of the present disclosure have determined that, contrary to the common general knowledge in the art, a key factor in successful deposition of medicament to the regions mentioned above is the force with which the combined propellant and medicament is delivered. A higher force tends to result in particular in a greater deposition of the medicament at the olfactory region. The high force contained in the spray will also result in sufficient penetration of spray thought he convoluted nasal cavity and nasal valve passages, leading to higher olfactory deposition. This finding is contrary to the teaching of the prior art discussed above, where low velocity is explicitly favoured, or achieved by means of e.g., a diffuser.

Introduction of the combined propellant and medicament into the nasal cavity at high velocity increases efficacy but can have certain drawbacks as will be discussed herein. The present disclosure seeks to ameliorate those drawbacks.

According to a first aspect of the disclosure there is provided a device for delivery of a medicament to at least one of the olfactory region, the turbinates region or the nasopharynx of a nasal cavity, the device comprising:
a canister comprising:
   a container containing a pressurised propellant and the medicament; and
   a metering valve configured to release a predetermined amount of the pressurised propellant and the medicament; and
an actuator comprising;
a housing receiving the canister;
   a valve block receiving the metering valve, the valve block defining an expansion chamber for passage of propellant and medicament expelled from the canister; and,
   a nosepiece for insertion into a user's nostril, the nosepiece comprising a fluid passage and a delivery opening for expulsion of the propellant and medicament into the user's nostril;
wherein a flow path is defined through the actuator for passage of propellant and medicament from the canister to the delivery opening, and wherein the cross-sectional area of the flow path is greater than the lowest cross-sectional area of the metering valve orifices at all points along the flow path.

Advantageously this decreased restriction provides for a higher flow rate and speed, which in turn results in a higher force of delivery. The applicant has identified that increased delivery force is a key parameter in delivery to the aforementioned deeper regions of the nasal cavity.

Preferably the metering valve comprises a valve stem having a valve stem inlet and a valve stem outlet, wherein the valve stem defines the lowest cross-sectional area of the metering valve.

Preferably the valve stem inlet has a cross-sectional area of at least 0.4 mm².

Preferably the valve stem inlet comprises a plurality of openings defined in a sidewall of the valve stem.

Preferably the plurality of openings define a total cross-sectional area of at least 0.4 mm².

The valve stem inlet may comprise a non-circular opening, for example a polygonal opening.

In one embodiment a flow path is defined through the actuator for passage of propellant and medicament from the canister to the delivery opening, and at least part of the flow path may be constructed from a conductive material having at least one of:
a thermal conductivity greater than or equal to 1 W/mK; or
an electrical resistivity less than or equal to 1×10¹⁰ Ω·m.

This combination results in a beneficial, synergistic effect because the increased delivery force of the second aspect has a risk of lower temperature delivery and/or higher deposition. Therefore the novel features of the first aspect help to mitigate this.

The delivery opening of the nosepiece may have a profile with a first dimension in a first direction and a second dimension in a second direction, wherein the first dimension is larger than the second dimension.

A single delivery tube may be provided extending from the valve block to the delivery opening.

The single delivery tube may extend from a position within the valve block to the delivery opening, and wherein the single delivery tube has an inlet facing the canister, and a curved portion directing the flow towards an outlet at the delivery opening.

The pressurised propellant may comprise at least 25% CO₂.

According to the first aspect there is also provided a method of administering a medicament to the interior surface of a nasal cavity comprising:
providing a device according to the first aspect;
inserting the nosepiece into the nostril of a human or animal; and
actuating the metering valve to release a bolus containing the medicament, during which actuation the nosepiece is held in position such that the bolus is ejected from the delivery opening into the nasal cavity.

Preferably the method is a method of treatment of a condition of the CNS in which the medicament is selected for a therapeutic effect via transmission to the CNS via the olfactory nerve.

According to a second aspect of the present disclosure there is provided a device for delivery of a medicament to at least one of the olfactory region, the turbinates region or the nasopharynx of a nasal cavity, the device comprising:
a canister comprising:
   a container containing a pressurised propellant and the medicament; and
   a metering valve configured to release a predetermined amount of the pressurised propellant and the medicament; and
an actuator comprising;
   a housing receiving the canister;
   a valve block receiving the metering valve, the valve block defining an expansion chamber for passage of propellant and medicament expelled from the canister; and
   a nosepiece for insertion into a user's nostril, the nosepiece comprising a fluid passage and a delivery opening for expulsion of the propellant and medicament into the user's nostril;
wherein a flow path is defined through the actuator for passage of propellant and medicament from the canister to the delivery opening, and wherein at least part of the flow path is constructed from a conductive material having at least one of:

a thermal conductivity greater than or equal to 1 W/mK; or
an electrical resistivity less than or equal to 1×10¹⁰ Ω·m.

Advantageously the use of such materials in the flow path provides an increased temperature of the fluid bolus which is more comfortable for the user. Reduced electrical resistivity allows electrostatic build up due to the passing fluid to be conducted away, which in turn reduces deposition and increases the amount of material exiting the device and entering the patient's nasal cavity.

The container contains the mixed propellant and medicament, which can be in a suspended or fully dissolved state i.e., can be a solution or a suspension formulation.

Preferably the at least part of the flow path constructed from a conductive material includes a portion of the flow path defined in the valve block.

Preferably the at least part of the flow path constructed from a conductive material includes at least a part of the expansion chamber.

The valve block may be constructed entirely from the conductive material.

Alternatively the valve block comprises a valve block body and an insert defining at least part of the flow path defined in the valve block, wherein the insert is constructed from the conductive material, and wherein the valve block body is constructed from a material having at least one of a lower thermal conductivity or higher electrical resistivity than the insert.

Preferably the insert is cylindrical in shape.

Preferably the insert abuts a first side of an internal flange of the valve block body, and wherein the metering valve abuts a second, opposite side of the flange of the valve block body.

Preferably a single uninterrupted flow path is defined through the nosepiece from the valve block body.

Preferably the single uninterrupted flow path is defined through a single delivery tube extending from the valve block body, through the nosepiece to the delivery opening.

The conductive material may be provided as a coating on an internal flow channel defining the flow path.

Preferably the single delivery tube extends partially into the valve block body.

The single delivery tube may follow a curved path at the valve block body to face in the direction of the canister.

Preferably wherein the conductive material comprises a metal, for example aluminium.

The cross-sectional area of the flow path may be greater than the lowest cross-sectional area of the metering valve at all points along the path.

The delivery opening of the nosepiece may have a profile with a first dimension in a first direction and a second dimension in a second direction, wherein the first dimension is larger than the second dimension.

A single delivery tube may extend from the valve block to the delivery opening.

The single delivery tube may extends from a position within the valve block to the delivery opening, and wherein the single delivery tube has an inlet facing the canister, and a curved portion directing the flow towards an outlet at the delivery opening.

The pressurised propellant may comprise at least 25% CO₂.

According to the second aspect there is also provided a method of administering a medicament to the interior surface of a nasal cavity comprising:
providing a device according to the second aspect;
inserting the nosepiece into the nostril of a human or animal;
actuating the metering valve to release a bolus containing the medicament, during which actuation the nosepiece is held in position such that the bolus is ejected from the delivery opening into the nasal cavity.

Preferably the method is a method of treatment of a condition of the CNS in which the medicament is selected for a therapeutic effect via transmission to the CNS via the olfactory nerve.

According to a third aspect of the disclosure there is provided a device for delivery of a medicament to at least one of the olfactory region, the turbinates region or the nasopharynx of a nasal cavity, the device comprising:
a canister comprising:
   a container containing a pressurised propellant and the medicament; and
   a metering valve configured to release a predetermined amount of the pressurised propellant and the medicament; and
an actuator comprising;
   a housing receiving the canister;
   a valve block receiving the metering valve, the valve block defining an expansion chamber for passage of propellant and medicament expelled from the canister; and
   a nosepiece for insertion into a user's nostril, the nosepiece comprising a fluid passage and a delivery opening for expulsion of the propellant and medicament into the user's nostril;
wherein the delivery opening has a profile with a first dimension in a first direction and a second dimension in a second direction, wherein the first dimension is larger than the second dimension.

Advantageously, the applicant has discovered that non-circular openings provide an increased force of expulsion, which is beneficial in application of the material to the deeper regions of the nasal cavity.

Preferably the delivery opening has a curved profile.

Preferably the delivery opening is elliptical.

Preferably a flow path is defined through the actuator for passage of propellant and medicament from the canister to the delivery opening, and wherein the cross-sectional area of the flow path transitions from a circular profile to the profile of the delivery opening.

A flow path may be defined through the actuator for passage of propellant and medicament from the canister to the delivery opening, and wherein the cross-sectional area of the flow path is greater than the lowest cross-sectional area of the metering valve at all points along the path.

Preferably the fluid passage of the nosepiece transitions from a circular profile to the profile of the delivery opening.

The third aspect may be combined with the second aspect such that in the third aspect,
a flow path is defined through the actuator for passage of propellant and medicament from the canister to the delivery opening, and wherein at least part of the flow path is constructed from a conductive material having at least one of:
a thermal conductivity greater than or equal to 1 W/mK; or
an electrical resistivity less than or equal to 1×10¹⁰ Ω·m.

This combination results in a beneficial, synergistic effect because the increased delivery force of the third aspect has a risk of lower temperature delivery. Therefore the novel features of the second aspect help to mitigate this.

It will be noted that any of the preferable or optional features of the second aspect may be combined with the third aspect, and any preferable or optional features thereof.

The third aspect may also be combined with the first aspect, such that in the third aspect a flow path is defined through the actuator for passage of propellant and medicament from the canister to the delivery opening, and wherein the cross-sectional area of the flow path is greater than the lowest cross-sectional area of the metering valve at all points along the path.

This combination results in a beneficial, synergistic effect. A substantial increase in delivery force is observed when combining the second and third aspects. Further, the first, second and third aspects may be combined such that temperature effects and/or deposition of the increased force is mitigated by the first aspect.

According to the third aspect there is also provided a method of administering a medicament to the interior surface of a nasal cavity comprising:
providing a device according to the third aspect;
inserting the nosepiece into the nostril of a human or animal;
actuating the metering valve to release a bolus containing the medicament, during which actuation the nosepiece is held in position such that the bolus is ejected from the delivery opening into the nasal cavity.

Preferably the method is a method of treatment of a condition of the CNS in which the medicament is selected for a therapeutic effect via transmission to the CNS via the olfactory nerve.

According to a fourth aspect there is provided a device for delivery of a medicament to at least one of the olfactory region, the turbinates region or the nasopharynx of a nasal cavity, the device comprising:
a canister comprising:
   a container containing a pressurised propellant and the medicament; and
   a metering valve configured to release a predetermined amount of the pressurised propellant and the medicament; and
an actuator comprising;
a housing receiving the canister;
a valve block receiving the metering valve; and
a nosepiece for insertion into a user's nostril, the nosepiece comprising a fluid passage and a delivery opening for expulsion of the propellant and medicament into the user's nostril;
a delivery tube extending from the valve block to the delivery opening;
wherein the delivery tube has an inlet facing in a first direction towards the canister, an outlet at the delivery opening facing in a second direction, such that the flow through the delivery tube changes direction from the first to the second end.

Advantageously, the single delivery tube both provides a smaller expansion chamber than the prior art, which acts to increase the velocity and force of delivery. Further, it allow the flow direction to change to the generally upward expulsion direction with minimal losses (through a curved path).

Preferably the delivery tube defines a curved portion.

Preferably the flow changes direction through more than 90 degrees.

Preferably the delivery tube is flexible.

Preferably the valve block defines a channel comprising an annular flange, wherein the metering valve abuts a first side of the annular flange, and wherein the first end of the delivery tube abuts a second side of the annular flange.

Preferably the delivery tube defines a constant cross-sectional area central passage.

Preferably a flow path is defined through the actuator for passage of propellant and medicament from the canister to the delivery opening, and wherein the cross-sectional area of the flow path is greater than the lowest cross-sectional area of the metering valve at all points along the path.

A flow path is defined through the actuator for passage of propellant and medicament from the canister to the delivery opening, and wherein at least part of the flow path may be constructed from a conductive material having at least one of:
a thermal conductivity greater than or equal to 1 W/mK; or
an electrical resistivity less than or equal to 1×10¹⁰ Ω·m.

The second end of the delivery tube may have a profile with a first dimension in a first direction and a second dimension in a second direction, wherein the first dimension is larger than the second dimension.

The pressurised propellant may comprise at least 25% CO₂.

According to the fourth aspect there is also provided a method of administering a medicament to the interior surface of a nasal cavity comprising:
providing a device according to the fourth aspect;
inserting the nosepiece into the nostril of a human or animal;
actuating the metering valve to release a bolus containing the medicament, during which actuation the nosepiece is held in position such that the bolus is ejected from the delivery opening into the nasal cavity.

Preferably the method is a method of treatment of a condition of the CNS in which the medicament is selected for a therapeutic effect via transmission to the CNS via the olfactory nerve.

According to a fifth aspect of the disclosure there is provided a device for delivery of a medicament to at least one of the olfactory region, the turbinates region or the nasopharynx of a nasal cavity, the device comprising:
a canister comprising:
   a container containing a pressurised propellant and the medicament; and
   a metering valve configured to release a predetermined amount of the pressurised propellant and the medicament; and
an actuator comprising;
   a housing receiving the canister;
   a valve block receiving the metering valve, the valve block defining an expansion chamber for passage of propellant and medicament expelled from the canister; and
   a nosepiece for insertion into a user's nostril, the nosepiece comprising a fluid passage and a delivery opening for expulsion of the propellant and medicament into the user's nostril;
   wherein the propellant comprises at least 25% CO₂.

Advantageously, the proposed propellant has a particular ability to generate a high expulsion force.

The fifth aspect may be combined with the first aspect such that a flow path is defined through the actuator for passage of propellant and medicament from the canister to the delivery opening, and wherein the cross-sectional area of the flow path is greater than the lowest cross-sectional area of the metering valve at all points along the path.

The fifth aspect may be combined with the second aspect such that a flow path is defined through the actuator for passage of propellant and medicament from the canister to the delivery opening, and wherein at least part of the flow path is constructed from a conductive material having at least one of:
a thermal conductivity greater than or equal to 1 W/mK; or
an electrical resistivity less than or equal to 1×10¹⁰ Ω·m.

The increased force provided by the novel propellants can cause a lower temperature and/or increased deposition which is mitigated by the first aspect.

The fifth aspect may be combined with the third aspect such that wherein the delivery opening has a profile with a first dimension in a first direction and a second dimension in a second direction, wherein the first dimension is larger than the second dimension.

The fifth aspect may be combined with the fourth aspect such that a single delivery tube extends from the valve block to the delivery opening.

The single delivery tube may extend from a position within the valve block to the delivery opening, and wherein the single delivery tube has an inlet facing the canister, and a curved portion directing the flow towards an outlet at the delivery opening.

The combination of the fifth aspect with the second or third aspects creates a much higher expulsion force, which is beneficial for N2B delivery.

It will be noted that the fifth aspect may be combined with combinations of the first, second, third or fourth aspects, or for optimum performance all five aspects may be combined.

According to the fifth aspect there is also provided a method of administering a medicament to the interior surface of a nasal cavity comprising:
providing a device according to the fifth aspect;
inserting the nosepiece into the nostril of a human or animal;
actuating the metering valve to release a bolus containing the medicament, during which actuation the nosepiece is held in position such that the bolus is ejected from the delivery opening into the nasal cavity.

Preferably the method is a method of treatment of a condition of the CNS in which the medicament is selected for a therapeutic effect via transmission to the CNS via the olfactory nerve.

The nosepiece of any aspect may be formed to aim the delivery outlet towards at least one of the turbinates region or the nasopharynx. The fluid passage of the nosepiece may have a first flow axis proximate the valve block, and a second flow axis proximate the delivery opening, wherein the first and second flow axes are at an angle to each other.

It will be noted that the present disclosure is suitable for use on humans as well as animals. The device may be adapted for adult or paediatric use.

All headings provided herein are for the convenience of the reader and should not be used to limit the meaning of any text that follows the heading, unless so specified.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements. The term “consisting of” means “including,” and is limited to whatever follows the phrase “consisting of.” Thus, the phrase “consisting of” indicates that the listed elements are required or mandatory and that no other elements may be present. The term “consisting essentially of” means including any elements listed after the phrase, and is limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The words “preferred” and “preferably” refer to embodiments of the disclosure that may afford certain benefits, under certain circumstances; however, other embodiments may also be preferred, under the same or other circumstances. Furthermore, the recitation of one or more preferred embodiments does not imply that other embodiments are not useful, and is not intended to exclude other embodiments from the scope of the disclosure.

In this application, terms such as “a,” “an,” and “the” are not intended to refer to only a singular entity, but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

These and other aspects of the present disclosure will be apparent from the detailed description below. In no event, however, should the above summaries be construed as limitations on the claimed subject matter, which subject matter is defined solely by the attached claims, as may be amended during prosecution.

### BRIEF DESCRIPTION OF DRAWINGS

Example devices according to the present disclosure will now be described with reference to the Figures in which:

FIG. 1 is a side section view of a first device according to the present disclosure;

FIG. 2 is a detailed side section view of a part of the device of FIG. 1;

FIG. 3a is a schematic view of a flow path of a known pMDI;

FIG. 3b is a schematic view of the flow path of the part the device of FIG. 2;

FIG. 4a is a detail view of a part of the device of FIG. 1;

FIG. 4b is a detail view of a part of a second device according to the present disclosure;

FIG. 4c is a detail view of a part of a third device according to the present disclosure;

FIG. 5 is a side section view of a fourth device according to the present disclosure;

FIG. 6 is a side section view of a fifth device according to the present disclosure;

FIGS. 7a to 7c are views of a component of a sixth device according to the present disclosure;

FIG. 8 is a side section view of a seventh device according to the present disclosure;

FIGS. 9a to 9e are views of various opening shapes;

FIGS. 10a to 10c are views of a component of an eighth device according to the present disclosure.

### DESCRIPTION OF EMBODIMENTS

### First Embodiment

### Configuration of the First Embodiment

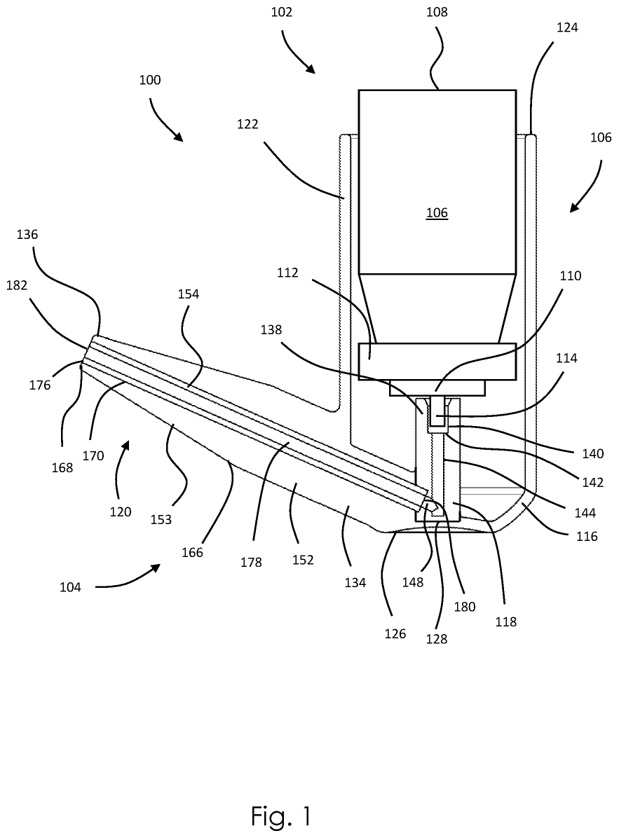

Referring to FIG. 1, a device 100 according to the disclosure in the form of a nasal pMDI is shown. The device 100 comprises a valved canister 102 and an actuator 104.

The canister 102 comprises a container 106 defining a base 108. At the opposite end of the base 108 there is provided a metering valve 110. The valve 110 is sealed to the container 106 via a crimp 112. The valve 110 comprises a valve stem 114 which is resiliently moveable towards the container 106. Upon depression of the valve stem 114 towards the container 106, the metering valve 110 is configured to release a predetermined (metered) volume of container fluid content from a valve orifice. Such valves are well known in the art.

The canister 102 is primarily constructed from a metal material (aluminium in this embodiment), although parts of the valve 110 are constructed from plastics materials, as is known it the art. Various coatings can be present on the internal surface of the canister as known in the art.

The container 106 contains a mixture of a propellant (in this embodiment HRC-134A, i.e., 1,1,1,2-Tetrafluoroethane) and a medicament formulation. In this embodiment the medicament is suitable for application to the olfactory region of the nasal canal to treat a condition of the CNS.

The actuator 104 comprises a housing 116, a valve block 118 and a delivery arrangement 120.

The housing 116 comprises a generally cylindrical canister-receiving portion 122 having an open end 124 and a closed end or base 126. The base 126 is generally circular and defines a valve block engaging recess 128 at its geometric centre.

Projecting radially outwardly from the cylindrical portion 122 and angled from the base 126 upwardly in the general direction of the open end 124 (i.e., projecting both radially and axially) is a nosepiece 152. The nosepiece 152 has a first end 134 in communication with the cylindrical portion 122 and a second, free end 136.

The valve block 118 is a unitary body defining an inlet 138 defining a valve stem receiving recess 140 having a shoulder 142. The recess 140 is in communication with an axial flow channel 144. Adjacent the flow channel 144 and in communication therewith, extending to a sidewall of the valve block 118 there is provided an exit opening 148.

The delivery arrangement 120 comprises the nosepiece 152 and a delivery tube 154.

The nosepiece 152 is elongate having an end portion 153 which is frustroconical in shape tapering from a wider first end 166 to a narrower second end 168. The nosepiece 152 defines a through bore 170 defining a delivery tube opening 176 at the second end 168. The through-bore is in fluid communication with the exit opening 148 of the valve block 118.

The delivery tube 154 is an elongate tube of constant cross section having a through-bore 178. It has an entry inlet 180 and a delivery outlet 182.

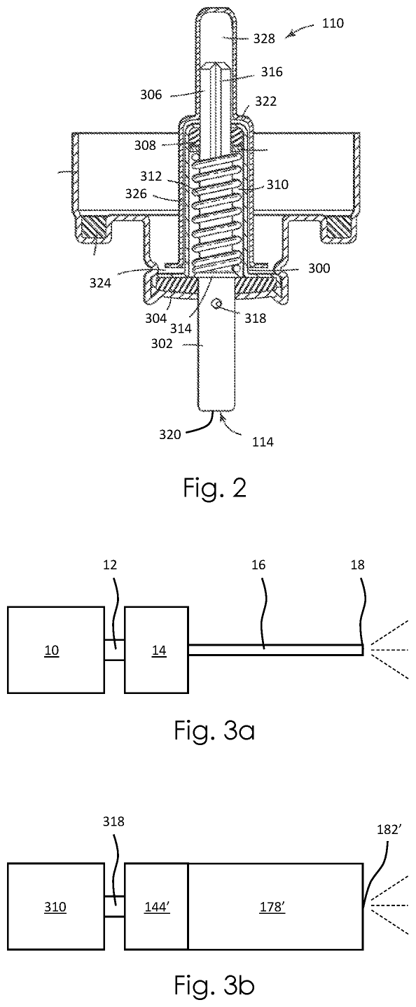

Referring to FIG. 2, the valve 110 is shown in more detail. The metering valve 110 comprises a valve stem 114 having an outlet 320 and a valve body 300. The valve stem 114 extends through a central aperture of the valve body 300. A lower stem portion 302 of the valve stem extends outwardly and is in slidable, sealing engagement with an outer seal 304 (also termed the diaphragm seal), while an upper stem portion 306 of the valve stem extends inwardly and is in slidable, sealing engagement with an inner seal 308 (also termed the metering gasket seal). A (non-transistory) metering chamber 310 is defined within the valve housing between the outer seal 304 and inner seal 308. A compression spring 312 is positioned within the valve housing with one end abutting the inner seal 308 and the other end abutting a flange 314 on the valve stem near the outer seal. Upon movement of the valve stem inwardly, a groove 316 in the upper stem portion 306 will pass beyond the inner seal 308 so that a complete seal is formed between the upper stem portion of the valve stem and the inner seal, thereby sealing off the metering chamber. Upon further movement of the valve stem inwardly, a valve stem inlet orifice 318 of the outlet passage of the valve stem passes the outer seal into the metering chamber and the contents of the metering chamber pass through the outlet passage of the valve stem, exiting the stem outlet 320. The valve 110 comprises a second valve body 322 defining a bottle emptier. When such a second valve body is provided, aerosol formulation in the container 106 will pass through a gap 324 between the first valve body 300 and the second valve body 322 (the gap 324 is near the outlet seal), through an annular gap 326 into a pre-metering region 328 and then through the groove 316 into the metering chamber 310.

### Assembly of the First Embodiment

The device 100 of the first embodiment is assembled as shown in FIG. 1. The actuator 104 is assembled by inserting the valve block 118 into the housing 116. The valve block may be secured with a mechanical (e.g., press or interference) fit, or with an adhesive. Once installed in this manner the valve stem receiving recess 140 faces upwardly towards the open end 124 of the housing 116.

The delivery tube 154 extends from valve block 118 to the free end of the nosepiece 152. The first end 180 is in fluid communication with the channel 144 of the valve block 118 (i.e., the expansion chamber). The second end 182 terminates at the tip of the nosepiece 152 at the delivery tube opening 176. Therefore the tube 154 provides an uninterrupted constant cross-section flow path from the expansion chamber in the valve block to the second end (i.e., the free end or tip) of the nosepiece.

The canister 102 is installed in the assembled actuator 104 by inserting the valve stem 114 into the valve stem receiving recess 140 such that it abuts the shoulder 142.

### Operation of the First Embodiment

During operation, the user inserts the nosepiece 152 into a nostril. A compressive force is applied with the user's hand between the base 108 of the canister 106 and the base 126 of the actuator 104. This opens the valve 110 which dispenses a bolus of mixed propellant and medicament under pressure into the flow channel 144. The flow channel 144 acts as an expansion chamber, allowing the compressed material to expand after exit from the canister 106. Upon reaching the opening 148 the flow changes direction to enter the bore 178 of the delivery tube 154 via the inlet 180. The flow then passes along the bore 178 to the second end and delivery outlet 182 where it exits the device 100 with such force and speed as to travel into the nasal cavity and contact the olfactory region of the nasal cavity. Transmission of the medicament via the N2B mechanism then occurs.

It will be noted that aside from the expansion within the flow channel there are no features of the flow path that cause an aerosolisation or ‘spread’ of the bolus. The delivery tube provides a single channel extending from the expansion chamber/sump to the top of the nosepiece without flow disruption. This provides a narrow, focussed stream of material contrary to many known pMDIs that deliberate seek to aerosolise the flow to provide a diffuse plume.

Referring to FIG. 3a, there is shown a schematic of a typical flow path from the metering chamber of the metering valve to the outlet in a prior art pMDI. Starting at the metering chamber 10, the flow passes through the valve stem orifice 12, to the expansion chamber 14 and to the spray orifice 16 and outlet 18. The spray orifice 16, not the valve orifice 12, is typically rate determining in terms of providing the main restriction to the flow. This spray orifice is typically the smallest orifice in the flow path (˜0.3-0.5 mm diameter), with the valve orifice being slightly larger (˜0.6 mm diameter).

Turning to FIG. 3b, the flow path according to the present embodiment is shown. The diameter of the bore 178 of the delivery tube 154 leading to the exit orifice 182 is configured to be larger than that of the valve stem orifice 318, and similar to that of the expansion chamber 144. In this embodiment, the restriction is moved from the spray orifice 16 to the valve orifice 318, which becomes the smallest cross-sectional area in the flow path.

The applicant has identified a relationship between spray force and spray orifice, i.e., that a larger orifice leads to a greater force. The reduced spray orifice restriction leads to the constant metered mass being expelled over a shorter time period, yielding greater force. The applicant has also discovered a positive correlation between plume force and olfactory deposition. This is associated with a lower pulmonary breakthrough. Therefore a larger orifice 182 provides better olfactory deposition in use. Evidently there is a requirement to restrict the flow as little as possible before the orifice. This is achieved by using a larger area bore 178, which moves the restriction to the valve orifice 318 as discussed above.

The effect is further enhanced by increasing the cross-sectional area of the valve stem orifice from prior art levels (˜0.6 mm diameter). This can be achieved by either increasing the diameter of the orifice:

| Flow path component | ID (mm) | C-S area (mm²) |
|---|---|---|
| Prior art | 0.635 | 0.3 |
| Novel spray orifice 1 | 0.75 | 0.4 |
| Novel spray orifice 2 | 1.60 | 2.0 |
| Novel spray orifice 3 | 3.00 | 7.1 |

or by increasing the number of bores in the stem 302 from one to several. For example:

| Valve orifice # | ID (mm) | Effective C-S area (mm²) |
|---|---|---|
| 1 (prior art) | 0.635 | 0.3 |
| 2 | 0.635 | 0.6 |
| 3 | 0.635 | 1.0 |
| 4 | 0.635 | 1.3 |
| 5 | 0.635 | 1.6 |
| 6 | 0.635 | 1.9 |

The following table combines the idea of an increased ID with increased number of valve stem orifices.

| Valve orifice # | ID (mm) | Effective C-S area (mm²) |
|---|---|---|
| 1 | 0.75 | 0.4 |
| 2 | 0.75 | 0.9 |
| 3 | 0.75 | 1.3 |
| 4 | 0.75 | 1.8 |
| 5 | 0.75 | 2.2 |
| 6 | 0.75 | 2.7 |
| 7 | 0.75 | 3.1 |
| 8 | 0.75 | 3.5 |
| 9 | 0.75 | 4.0 |
| 10 | 0.75 | 4.4 |
| 11 | 0.75 | 4.9 |
| 12 | 0.75 | 5.3 |

Significant benefit is seen by the combination of increasing the effective valve orifice area (i.e., a larger orifice or the total area if multiple orifices are used) and configuring the device such that the valve orifice is the flow restriction from the metering chamber to the outlet.

It will be noted that the valve stem outlet 320 is typically 1.6 mm in diameter and as such any increase above this (in this case an effective area of 2.0 mm²) is unlikely to be effective unless the valve stem itself is increased in diameter (which would enable the outlet 320 to be enlarged). Increasing the valve stem diameter would also provide more space to enable the higher number of orifices to be positioned on the sidewall.

The multiple orifices may be positioned on a single circumferential line, or on two or more offset circumferential lines around the stem.

### Variations on the First Embodiment

In one further embodiment, the outer and inner diameter of the valve stem may be increased which would provide more scope for a larger effective cross-section for the valve stem orifice and the valve stem outlet.

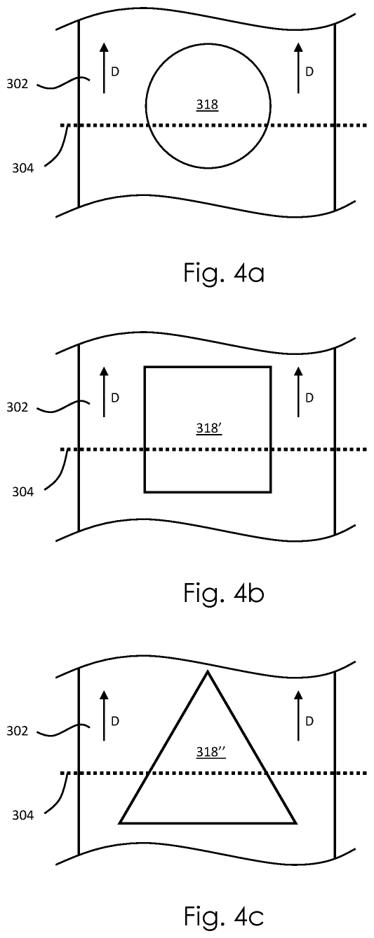

In another variation, the circular valve stem orifice(s) may be replaced with a polygonal or partially polygonal valve stem orifice. Referring to FIG. 4a the circular orifice 318 is shown, with the top of the seal 304 indicating the line past which fluid can exit the valve upon depression D. In FIG. 4a, the area of the orifice exposed to the chamber does not increase linearly. In FIG. 4b, with a polygonal, or part polygonal orifice 318 the width of the orifice does not vary in the direction of travel of the stem. Therefore, flow area increases linearly. It will also be noted that for a given width and height, a square or rectangular orifice is a more efficient use of space—i.e., the equivalent area is greater.

FIG. 4c shows a further embodiment with a triangular orifice 318″ in which the widest part (base) of the triangle is exposed first.

Non-circular holes may be formed by e.g., laser drilling.

### Second Embodiment

### Configuration of the Second Embodiment

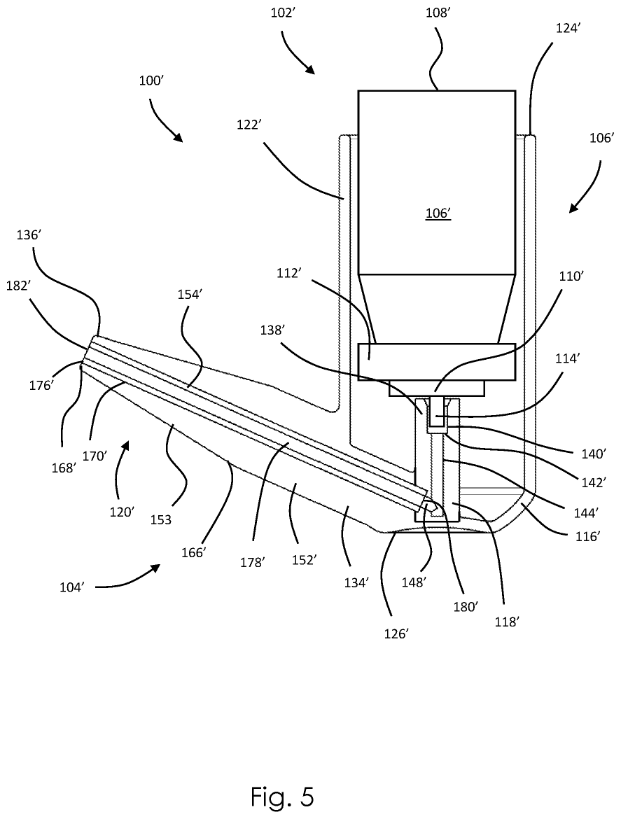

Referring to FIG. 5, there is shown a pMDI 100′ similar to the first embodiment. Similar features will be referred to using the prime suffix (e.g., 100′, 102′ etc).

As with the first embodiment, the valve block 118′ is a unitary body defining an inlet 138′ defining a valve stem receiving recess 140′ having a shoulder 142′. The recess 140′ is in communication with an axial flow channel 144′ leading to an outlet orifice 148′.

In contrast to traditional devices of this type the valve block 118′ is constructed from a metal material. In this embodiment the material is aluminium (although other metals are envisaged).

In order to reach the olfactory region of the nasal cavity, the bolus is projected at high speed and force. The construction of the valve block 118′ from a metal material has several beneficial effects from this point of view.

Firstly, the bolus tends to reduce in temperature as it expands in volume exiting the canister, i.e., within the flow channel 144′ acting as an expansion chamber. Ejection of a low temperature bolus into the nasal cavity can cause significant discomfort to the user, and this is exacerbated by the fact that in the present disclosure delivery via the N2B route requires a concentrated ‘jet’ of material (rather than a diffuse plume) directed at high speed (to reach the olfactory region). Use of a metal valve block 118′ allows thermal conduction to take place from the device's surroundings to raise the temperature of the bolus as it expands in, and passes through, the channel 144′. This increases user comfort.

Traditional plastics materials typically have a very low thermal conductivity. Metals tend to be much higher as exemplified in the table below. Thermally conductive plastics also work, and have the advantage of being less dense (lighter) and more readily mouldable. Increased conductivity can be achieved by inclusion of proprietary additive materials in a composite matrix with the base polymer, e.g., polypropylene in the table below:

| | Thermal conductivity |
|---|---|
| Material | [W/(m K)] |
| Polytetrafluoroethylene | 0.25 |
| Polycarbonate | 0.19 |
| Polyoxymethylene (Delrin/Acetal) | 0.23 |
| Polypropylene | 0.1-0.22 |
| Polypropylene (Thermally conductive) | 1.1-5.0 |
| Steel | 16 |
| Aluminum | 205 |
| Copper | 401 |

A further problem with prior art plastic components is the tendency for deposition to occur. This may occur through a number of mechanisms, for example friction causing a static electrical charge to occur on the surface of the plastic. This in turn can cause deposition of material onto the components, inhibiting ejection of the full dose. The applicants have discovered that use of a metal valve block 118 increases the amount of medicament delivered and as such the ability of the metal material to disperse any electrical charge is an advantage.

It will be noted that as well as metals, other materials exhibiting a thermal conductivity greater than or equal to 1 W/mK at ambient conditions (room temperature/pressure) are suitable. These include metals other than those examples above, alloys, alternate plastics and ceramics.

Traditional plastics materials typically have a very high electrical resistivity (and therefore a low conductivity). Metals and plastics doped with conductive additives such as carbon black tend to be lower as exemplified in the table below:

| Material | Electrical resistivity - Ω · m |
|---|---|
| Polycarbonate | 1 × 10¹⁴ |
| Polypropylene | 1 × 10¹³ |
| Polyoxymethylene (Delrin/Acetal) | 1 × 10¹² |
| Polypropylene - antistatic | 1 × 10¹⁰ |
| Polycarbonate - conductive | 1 |
| Steel | 2 × 10⁻⁷ |
| Aluminum | 3 × 10⁻⁸ |
| Copper | 2 × 10⁻⁸ |

It will be noted that as well as metals, other materials exhibiting a resistivity less than or equal to 1×10¹⁰ Ω·m at ambient conditions (room temperature/pressure) are suitable. These include metals other than those examples above, alloys, alternate plastics and ceramics.

These effects are particularly acute when the valve block 118′ is constructed from a thermally and electrically conductive material, such as a metal. This is because the valve block carries the material when it is as its highest speed and lowest temperature. The former factor provides a greater mitigation of deposition and the latter a greater increase in temperature (due to the high temperature differential between the material and its surroundings). The effect is also seen due to the passage of the material through an elongate constant cross section channel (the axial flow channel 144′).

### Third Embodiment

### Configuration of the Third Embodiment

The third embodiment is very similar to the second embodiment. The only difference is the valve block.

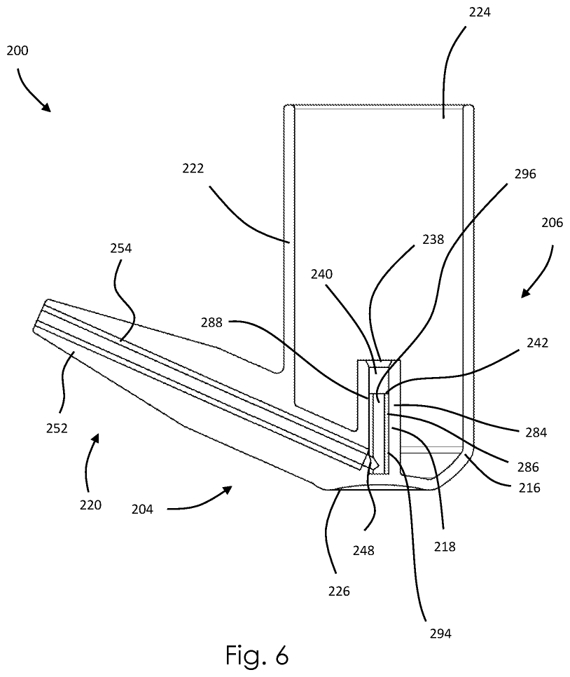

Referring to FIG. 6, a device 200 according to the disclosure in the form of a nasal pMDI is shown. The device 200 comprises a valved canister (not visible) and an actuator 204.

The actuator 204 comprises a housing 216, a valve block 218 and a delivery arrangement 220.

As with the second embodiment, the housing 216 comprises a generally cylindrical canister-receiving portion 222 having an open end 224 and a closed end or base 226. The base 226 is generally circular.

The valve block 218 comprises a valve block body 284 unitary with the housing 216 and a valve block insert 286.

The valve block body 284 defines an inlet 238 defining a valve stem receiving recess 240 having a shoulder 242. An axial bore 288 is provided through the body leading to an exit orifice 248. In contrast to the second embodiment, the valve block body 284 is constructed from a plastics material.

The valve block insert 286 is a tube defining a cylindrical outer surface 294 and an axial flow channel 296. The valve block insert is constructed from a metal material, in this embodiment aluminium.

As with the first embodiment, the delivery assembly 220 comprises a nosepiece 252 and a delivery tube 254.

### Operation of the Third Embodiment

During operation, the user inserts the nosepiece 252 into a nostril. A compressive force is applied with the user's hand between the base of the canister and the base 226 of the actuator 204. This opens the valve which dispenses a bolus of mixed propellant and medicament under pressure into the flow channel 296 of the insert 286.

Upon exiting the insert 286, the flow changes direction to enter the delivery tube 254. The flow then passes along the tube 254 to a delivery outlet where it exits the device 200 with such force and speed as to travel into the nasal cavity and contact the olfactory region of the nasal cavity. Transmission of the medicament via the N2B mechanism then occurs.

As with the second embodiment, the provision of the flow channel 296 with a metal surface is beneficial in terms of both the temperature of the bolus, and also reducing deposition within the device 200.

The applicant has determined that for stem blocks with an aluminium flow path (per the above embodiments), beneficial effects are seen in reductions in device retention (i.e., how much of the propelled substance is retained after use), reductions in electrostatic potential and increases in temperature of the ejected material.

### Variations on the Second and Third Embodiments

The material for the valve block or valve block insert may be selected due to only one of thermally and electrically conductive properties.

It will be noted that aside from the valve block, other components forming the flow path from the canister to the outlet (i.e., in direct contact with the passing fluid material) may be constructed from electrically and thermally conductive materials.

Other components defining the flow path from the canister to the opening at the tip of the nosepiece may be constructed from conductive materials. For example, the delivery tube 154 may be constructed from a conductive material such as metal.

It is also possible to coat the interior surfaces of the channels and openings with conductive material in a further embodiment. What is important is that the conductive material is in contact with the fluid within the channel.

The conductive material channel features of the second and third embodiments may be combined with the features of the first embodiment (i.e., increase cross sectional area of flow restriction). It will be noted that the first embodiment has the effect of increasing the force with which the bolus is expelled. This exacerbates the temperature reduction, or ‘cold’ feel by the user. Therefore, combining the features of the first embodiment with the second or third offers a synergistic effect—i.e., one of the problems associated with high force expulsion can be partially mitigated.

### Fourth Embodiment

### Configuration of the Fourth Embodiment

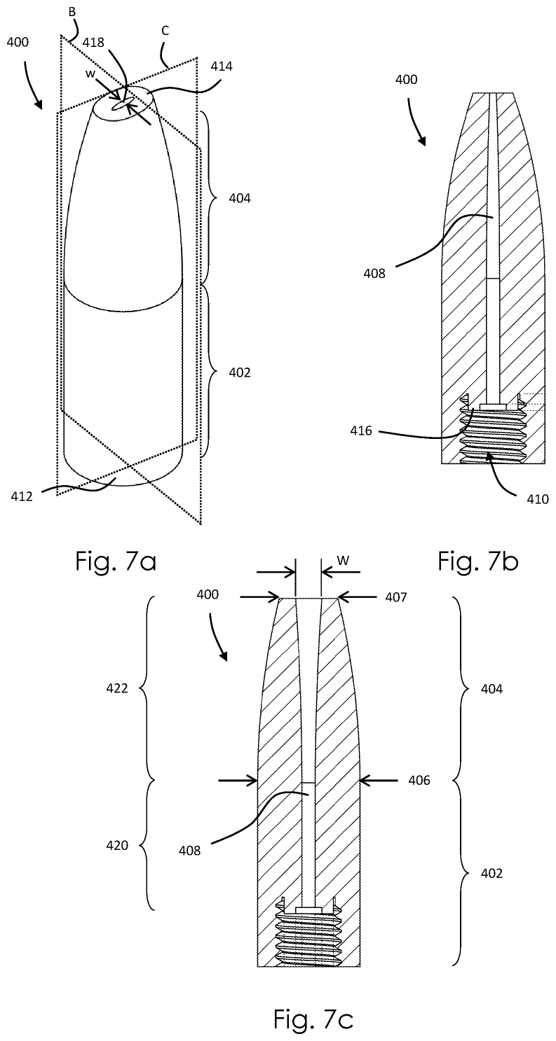

Referring to FIGS. 7a to 7c there is shown a nosepiece 400 according to the disclosure for use with any of the first to third embodiments, or variations thereof. The nosepiece 400 is an alternative to the nosepieces 152, 252. The nosepiece 400 has a first end 412 and a second end 414. It is elongate and comprises a cylindrical portion 402 and a frustroconical portion 404, tapering from a wider cross section 406 at the cylindrical portion 402 to a narrower cross section 407 at the second end 414.

The nosepiece 400 defines a through bore 408 and a mouth 410 at the first end 412 defining an internal shoulder/valve seat 416. The through bore 408 defines a delivery tube opening 418 at the second end 414. The delivery tube opening 418 is elliptical having a major width W and a minor width w. The major width W is at least 10% greater than the minor width w, i.e., W≥1.1w.

The through bore 408 comprises a first, circular cross-section, portion 420 and a second portion 422 leading to the opening 418. The second portion 422 is tapered and gradually transitions from the circular cross section of the first portion 420 to the elliptical opening 418.

The applicant has realised that an increase in delivery force occurs with the adoption of an elliptical nozzle. Further, compared to a traditional circular orifice, the spray pattern area is reduced, which provides for more targeted deposition (i.e., at the olfactory region).

### Operation of the Fourth Embodiment

The applicant has found that there is a surprising increase in spray force produced by an elliptical nozzle compared to a circular nozzle.

### Variations on the Fourth Embodiment

The ratio of W:w may vary, and may be, e.g., 2:1 or up to 16:1.

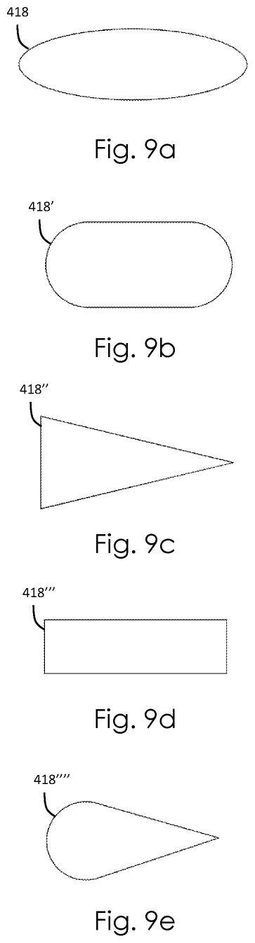

In addition to elliptical nozzles, other non-circular or asymmetric shapes may be used to provide the same effect. For example rectangular ‘postbox’ nozzles work, in addition to ‘teardrop’ or tapered nozzle shapes. Turning to FIGS. 9a to 9e, examples are shown. FIG. 9a shows the elliptical opening 418. FIG. 9b shows an obround or slot opening 418′. FIG. 9c shows a triangular opening 418″. FIG. 9d shows a rectangular or letterbox shape 418″. FIG. 9e shows a teardrop shape 418″″.

### Fifth Embodiment

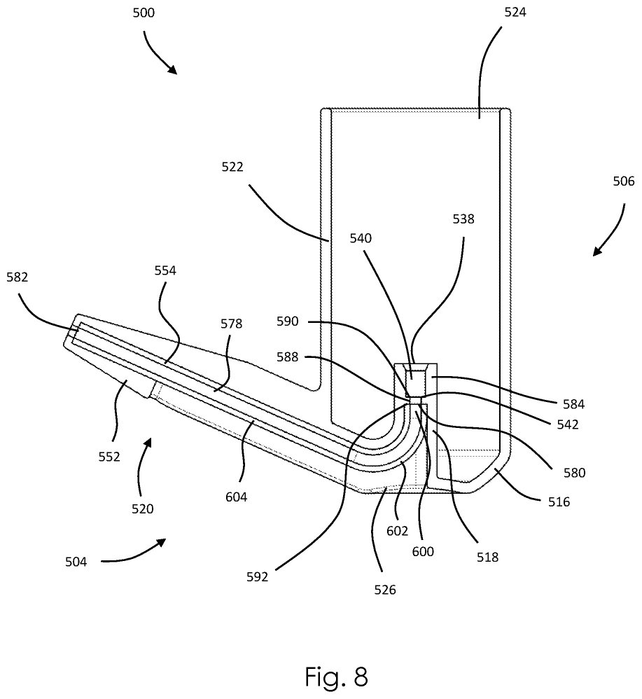

Referring to FIG. 8 there is shown a fifth embodiment. Referring to FIG. 8, a device 500 according to the disclosure in the form of a nasal pMDI is shown. The device 500 comprises a valved canister (not visible) and an actuator 504.

The actuator 504 comprises a housing 516 comprising an integral valve block 518 and a delivery arrangement 520.

As with the first embodiment, the housing 516 comprises a generally cylindrical canister-receiving portion 522 having an open end 524 and a closed end or base 526.

The valve block body 584 body is formed integrally with the housing 516 and defines an inlet 538 defining a valve stem receiving recess 540 having a shoulder 542. An axial bore 588 is provided through the body. An internal annular flange 590 is provided within the channel adjacent the recess 540 and defining the shoulder 542 of the recess 540 on one side, and an insert abutment shoulder 592 on a second side. In contrast to the second embodiment, the valve block body 584 is constructed from a plastics material.

The delivery assembly 520 comprises a nosepiece 552 integral with the housing 516 and a delivery tube 554.

The delivery tube 554 is an elongate tube of constant cross section having a through-bore 578. It has an entry inlet 580 and a delivery outlet 582. The delivery tube 554 has a first, straight portion 600 extending from the inlet 580 to a curved portion 602. The curved portion has a radius and redirects the flow through >90 degrees to a second straight portion 604 and ultimately to the outlet 582. The inlet 580 abuts the underside of the annular flange 590 and thus receives the bolus of material directly from the canister. The delivery tube therefore extends into the valve block 518 such that the inlet faces upwards, towards the canister, aligned and co-axial with the valve stem receiving recess 540.

### Operation of the Fifth Embodiment

The fifth embodiment is operated in the same way as the first embodiment. During use, the main difference is that the fact that the flow passes quickly from the canister to the delivery tube 554. The expansion chamber is therefore much smaller and defined within the delivery tube instead of the valve block. The reduced volume expansion chamber acts to reduce evaporation leading to a reduced vapour fraction and greater force of delivery.

### Sixth Embodiment

The above embodiments utilise the propellant HRC-134A. This propellant is known in the art for use within pMDIs. Other propellants may be used such as HRC-134A- these are HFA-227 (1,1,1,2,3,3,3-Heptafluoropropane) and HFA-152a (1,1-Difluoroethane). As discussed, the unique technical effect that the applicant has achieved with the present disclosure is the increase in the force of expulsion of the bolus of material from the subject device in order to impact the olfactory region of the nasal cavity

In order to achieve this effect, the applicant has identified an alternative propellant that provide a surprising increase in force and therefore efficacy of pMDIs when used for N2B delivery.

In this embodiment, a device according to any of the first to the fifth embodiments or variations thereof is used with a carbon dioxide (CO₂) based propellant. The applicant has discovered that the used of carbon dioxide as a propellant increases the force at which the mixed propellant/medicament is expelled.

Although replacement of known propellants with CO₂ in known nasal pMDIs increases the delivery force and effectiveness of N2B delivery, there is a marked synergistic effect between the use of CO₂ as a propellant and the used of any of the novel features of any of the above embodiments alone or in combination.

### Variations on the Sixth Embodiment

As well as 100% CO₂ propellant, propellants containing at least 25% CO₂ by weight are effective. The remaining propellant is a known propellant e.g., HRC-134A or another propellant discussed above.

### Seventh Embodiment

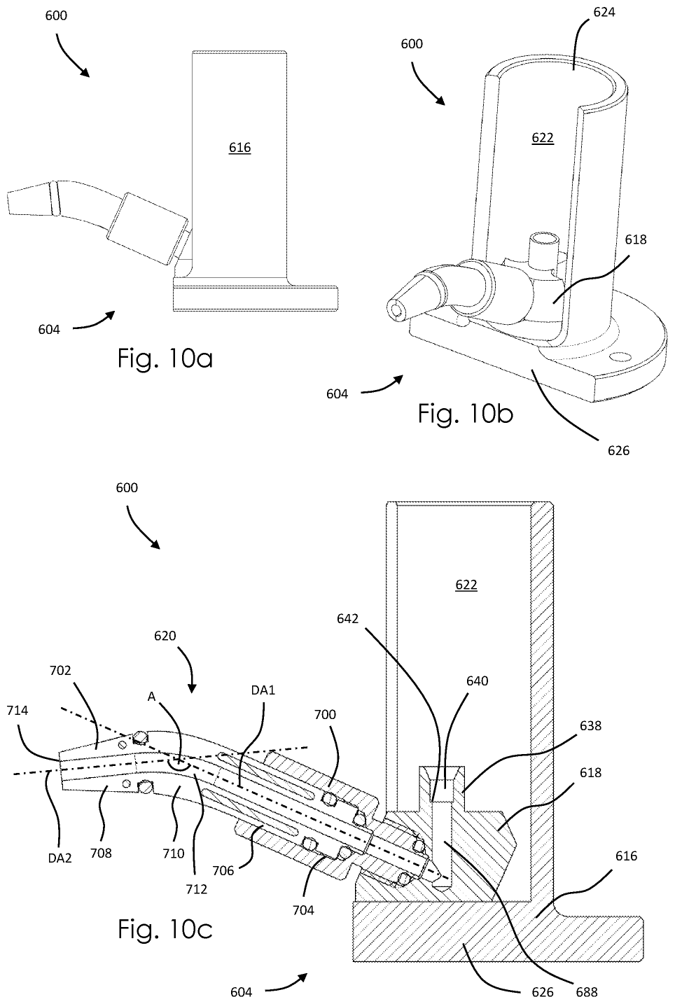

Referring to FIGS. 10a to 10c there is shown a fifth embodiment. Referring to FIG. 10a, a device 600 according to the disclosure in the form of a nasal pMDI is shown. The device 500 comprises a valved canister (not visible) and an actuator 604. It will be noted that the actuator shown 604 is of a preliminary design, rather than a production design, but essentially it demonstrates the operation of the disclosure in the same manner as a production device.

The actuator 604 comprises a housing 616 with a valve block 618 and a delivery arrangement 620.

As with the first embodiment, the housing 616 comprises a generally cylindrical canister-receiving portion 622 having an open end 624 and a closed end or base 626.

The valve block 618 defines an inlet 638 defining a valve stem receiving recess 640 having a shoulder 642. An axial bore 688 is provided through the body.

The delivery assembly 620 comprises a body 700 and a nosepiece 702. The body 700 is straight and defines a first delivery axis DA1 on which is centred a through bore 704. The nosepiece 702 comprises a tubular body having a proximal section 706, a distal section 708 and an intermediate section 710 (NB “distal” and “proximal” are used with reference to the device, not the user). The nosepiece 702 has a central through bore 712 extending to a delivery outlet 714. The proximal section 706 is partially inserted into the body 700 so as to be in fluid communication therewith. The proximal section 706 is straight and aligned with the first delivery axis DA1. The intermediate section 710 is curved, and the distal section 708 is straight, aligned in a second delivery axis DA2 that is angled to DA1 by angle A. In this embodiment the angle A is 150 degrees such that the distal section 708 is angled downwards with respect to the proximal section 706.

The body 700 is installed in the valve block 618 such that it is in fluid communication with the axial bore 688.

### Operation of the Seventh Embodiment

The seventh embodiment is operated in the same way as the first embodiment. During use, the main difference is that the fact that the flow is redirected through the nosepiece 702 such that instead of being directed to the olfactory region, it is directed to the nasopharynx. It will be noted that the seventh embodiment is combined with one or more of the first to sixth embodiments in order to increase the delivery force such that the medicament can reach the nasopharynx.

### Variations on the Sixth Embodiment

The geometry of the nosepiece may be modified to reach the turbinates region rather than the nasopharynx. In this instance, the nosepiece would be angled laterally.

### Combinations of the Embodiments

Each of the above embodiments provides a marked improvement in N2B delivery via the increase in particle speed and average flow density. This increases the delivery force, and the applicant has identified that increased force provides increased contact with the olfactory region.

It will be noted that combinations of the above embodiments provide marked synergistic effects above a simple combination of the individual contributions. For example:
The first embodiment (wider upstream flow restriction) can be enhanced by implementing either the second or third embodiments (conductive flow channel) because the lower temperature caused by the expansion and flow speed of the former can be mitigated by the latter;
The first embodiment (wider upstream flow restriction) can be enhanced by implementing the fourth embodiment (elliptical nozzle) to create a significantly improved/increased force of expulsion;
The first embodiment (wider upstream flow restriction) can be enhanced by implementing the fifth embodiment (delivery tube to canister) to create a significantly improved/increased force of expulsion;
The first embodiment (wider upstream flow restriction) can be enhanced by implementing the sixth embodiment (CO₂ propellant) to create a significantly improved/increased force of expulsion;
The second or third embodiments (conductive flow channel) can also be used to mitigate the lower flow temperature of any, some or all of the fourth (elliptical), fifth (tube) or sixth (CO₂) embodiments as well;
The fourth embodiment (elliptical nozzle) can be combined with the fifth (tube) and/or sixth (CO₂) embodiments to provide increased flow force.
All of the embodiments (including only one of the second and third embodiments) can also be combined to provide a greater increase in performance.

### Illustrative Aspects

The following are illustrative aspects of the disclosure.

In independent aspect A1, a device for delivery of a medicament to at least one of the olfactory region, the turbinates region and the nasopharynx of a nasal cavity as described herein comprising: a canister comprising: a container containing a pressurised propellant and the medicament; and a metering valve configured to release a predetermined amount of the pressurised propellant and the medicament; and an actuator comprising: a housing receiving the canister; a valve block receiving the metering valve, the valve block defining an expansion chamber for passage of propellant and medicament expelled from the canister; and a nosepiece for insertion into a user's nostril, the nosepiece comprising a fluid passage and a delivery opening for expulsion of the propellant and medicament into the user's nostril; wherein a flow path is defined through the actuator for passage of propellant and medicament from the canister, the flow path including the expansion chamber, fluid passage and the delivery opening, and wherein the cross-sectional area of the flow path is greater than the lowest cross sectional area of the metering valve at all points along the flow path.

In aspect A2 according to aspect A1, the metering valve comprises a valve stem having a valve stem inlet and a valve stem outlet, wherein the valve stem defines the lowest cross-sectional area of the metering valve.

In aspect A3 according to aspect A2, the valve stem inlet has a cross-sectional area of at least 0.4 mm².

In aspect A4 according to any of aspects A2 to A3, the valve stem inlet comprises a plurality of openings defined in a sidewall of the valve stem.

In aspect A5 according to aspect A4, the plurality of openings define a total cross-sectional area of at least 0.4 mm².

In aspect A6 according to any of aspects A2 to A5, the valve stem inlet comprises a non-circular opening.

In aspect A7 according to aspect A6, the valve stem inlet comprises a polygonal opening.

In aspect A8 according to any preceding aspect, at least part of the flow path is constructed from a conductive material having at least one of: a thermal conductivity greater than or equal to 1 W/mK; and an electrical resistivity less than or equal to 1×10¹⁰ Ω·m.

In aspect A9 according to any preceding aspect, the delivery opening of the nosepiece has a profile with a first dimension in a first direction and a second dimension in a second direction, wherein the first dimension is larger than the second dimension.

In aspect A10 according to any preceding aspect, further comprising a single delivery tube extending from the valve block to the delivery opening.

In aspect A11 according to aspect A10, the single delivery tube extends from a position within the valve block to the delivery opening, and wherein the single delivery tube has an inlet facing the canister, and a curved portion directing the flow towards an outlet at the delivery opening.

In aspect A12 according to any preceding aspect, the pressurised propellant comprises at least 25% CO₂.

In aspect A13 according to any preceding aspect, the nosepiece is formed to aim the delivery outlet towards at least one of the turbinates region and the nasopharynx.

In aspect A14 according to aspect A13, the fluid passage of the nosepiece has a first flow axis proximate the valve block, and a second flow axis proximate the delivery opening, wherein the first and second flow axes are at an angle to each other.

In independent aspect B1, a device for delivery of a medicament to at least one of the olfactory region, the turbinates region and the nasopharynx of a nasal cavity as described herein comprising: a canister comprising: a container containing a pressurised propellant and the medicament; and a metering valve configured to release a predetermined amount of the pressurised propellant and the medicament; and an actuator comprising: a housing receiving the canister; a valve block receiving the metering valve, the valve block defining an expansion chamber for passage of propellant and medicament expelled from the canister; and a nosepiece for insertion into a user's nostril, the nosepiece comprising a fluid passage and a delivery opening for expulsion of the propellant and medicament into the user's nostril; wherein a flow path is defined through the actuator for passage of propellant and medicament from the canister, the flow path including the expansion chamber, fluid passage and the delivery opening, and wherein at least part of the flow path is constructed from a conductive material having at least one of: a thermal conductivity greater than or equal to 1 W/mK; and an electrical resistivity less than or equal to 1×10¹⁰ Ω·m.

In aspect B2 according to aspect B1, the at least part of the flow path constructed from a conductive material includes a portion of the flow path defined in the valve block.

In aspect B3 according to aspect B1, the at least part of the flow path constructed from a conductive material includes at least a part of the expansion chamber.

In aspect B4 according to aspect B2 or B3, the valve block is constructed entirely from the conductive material.

In aspect B5 according to aspect B2 or B3, the valve block comprises a valve block body and an insert defining at least part of the flow path defined in the valve block, wherein the insert is constructed from the conductive material, and wherein the valve block body is constructed from a material having at least one of a lower thermal conductivity and higher electrical resistivity than the insert.

In aspect B6 according to aspect B5, the insert is cylindrical in shape.

In aspect B7 according to aspect B5 or B6, the insert abuts a first side of an internal flange of the valve block body, and wherein the metering valve abuts a second, opposite side of the flange of the valve block body.

In aspect B8 according to any of aspects B1 to B7, a single uninterrupted flow path is defined through the nosepiece from the valve block body.

In aspect B9 according to aspect B8, the single uninterrupted flow path is defined through a single delivery tube extending from the valve block body, through the nosepiece to the delivery opening.

In aspect B10 according to any of aspects B1 to B9, the conductive material is provided as a coating on an internal flow channel defining the flow path.

In aspect B11 according to any of aspects B1 to B10, the conductive material comprises a metal.

In aspect B12 according to aspect B11, the conductive material comprises aluminium.

In aspect B13 according to any of aspects B1 to B12, the cross-sectional area of the flow path is greater than the lowest cross-sectional area of the metering valve at all points along the path.

In aspect B14 according to any of aspects B1 to B13, the delivery opening of the nosepiece has a profile with a first dimension in a first direction and a second dimension in a second direction, wherein the first dimension is larger than the second dimension.

In aspect B15 according to any of aspects B1 to B14, comprising a single delivery tube extending from the valve block to the delivery opening.

In aspect B16 according to aspect B15, the single delivery tube extends from a position within the valve block to the delivery opening, and wherein the single delivery tube has an inlet facing the canister, and a curved portion directing the flow towards an outlet at the delivery opening.

In aspect B17 according to any of aspects B1 to B16, the pressurised propellant comprises at least 25% CO₂.

In aspect B18 according to any of aspects B1 to B17, the nosepiece is formed to aim the delivery outlet towards at least one of the turbinates region and the nasopharynx.

In aspect B19 according to aspect B18, the fluid passage of the nosepiece has a first flow axis proximate the valve block, and a second flow axis proximate the delivery opening, wherein the first and second flow axes are at an angle to each other.

In independent aspect C1, a device for delivery of a medicament to at one of the olfactory region, the turbinates region and the nasopharynx of a nasal cavity as described herein comprising: a canister comprising: a container containing a pressurised propellant and the medicament; and a metering valve configured to release a predetermined amount of the pressurised propellant and the medicament; and an actuator comprising; a housing receiving the canister; a valve block receiving the metering valve, the valve block defining an expansion chamber for passage of propellant and medicament expelled from the canister; and a nosepiece for insertion into a user's nostril, the nosepiece comprising a fluid passage and a delivery opening for expulsion of the propellant and medicament into the user's nostril; wherein the delivery opening has a profile with a first dimension in a first direction and a second dimension in a second direction, wherein the first dimension is larger than the second dimension.

In aspect C2 according to aspect C1, the delivery opening has a curved profile.

In aspect C3 according to aspect C1, the delivery opening is elliptical.

In aspect C4 according to any of aspects C1 to C3, a flow path is defined through the actuator for passage of propellant and medicament from the canister to the delivery opening, and wherein the cross-sectional area of the flow path transitions from a circular profile to the profile of the delivery opening.

In aspect C5 according to aspect C4, the fluid passage of the nosepiece transitions from a circular profile to the profile of the delivery opening.

In aspect C6 according to any of aspects C1 to C5, a flow path is defined through the actuator for passage of propellant and medicament from the canister to the delivery opening, and wherein the cross-sectional area of the flow path is greater than the lowest cross-sectional area of the metering valve at all points along the path.

In aspect C7 according to any of aspects C1 to C6, a flow path is defined through the actuator for passage of propellant and medicament from the canister, the flow path including the expansion chamber, fluid passage and the delivery opening, and wherein at least part of the flow path is constructed from a conductive material having at least one of: a thermal conductivity greater than or equal to 1 W/mK; and an electrical resistivity less than or equal to 1×10¹⁰ Ω·m.

In aspect C8 according to any of aspects C1 to C7, comprising a single delivery tube extending from the valve block to the delivery opening.

In aspect C9 according to aspect C8, the single delivery tube extends from a position within the valve block to the delivery opening, and wherein the single delivery tube has an inlet facing the canister, and a curved portion directing the flow towards an outlet at the delivery opening.

In aspect C10 according to any of aspects C1 to C9, the pressurised propellant comprises at least 25% CO₂.

In aspect C11 according to any of aspects C1 to C10, the nosepiece is formed to aim the delivery outlet towards at least one of the turbinates region and the nasopharynx.

In aspect C12 according to aspect C11, the fluid passage of the nosepiece has a first flow axis proximate the valve block, and a second flow axis proximate the delivery opening, wherein the first and second flow axes are at an angle to each other.

In independent aspect D1, a device for delivery of a medicament to at least one of the olfactory region, the turbinates region and the nasopharynx of a nasal cavity as described herein comprising: a canister comprising: a container containing a pressurised propellant and the medicament; and a metering valve configured to release a predetermined amount of the pressurised propellant and the medicament; and an actuator comprising: a housing receiving the canister; a valve block receiving the metering valve; and a nosepiece for insertion into a user's nostril, the nosepiece comprising a fluid passage and a delivery opening for expulsion of the propellant and medicament into the user's nostril; a delivery tube extending from the valve block to the delivery opening; wherein the delivery tube has an inlet facing in a first direction towards the canister, an outlet at the delivery opening facing in a second direction, such that the flow through the delivery tube changes direction from the first to the second end.

In aspect D2 according to aspect D1, the delivery tube defines a curved portion.

In aspect D3 according to aspect D1 or D2, the flow changes direction through more than 90 degrees.

In aspect D4 according to any of aspects D1 to D3, the delivery tube is flexible.

In aspect D5 according to any of aspects D1 to D4, the valve block defines a channel comprising an annular flange, wherein the metering valve abuts a first side of the annular flange, and wherein the first end of the delivery tube abuts a second side of the annular flange.

In aspect D6 according to any of aspects D1 to D5, the delivery tube defines a constant cross-sectional area central passage.

In aspect D7 according to any of aspects D1 to D6, a flow path is defined through the actuator for passage of propellant and medicament from the canister the flow path including the expansion chamber, fluid passage and the delivery opening, and wherein the cross-sectional area of the flow path is greater than the lowest cross-sectional area of the metering valve at all points along the path.

In aspect D8 according to any of aspects D1 to D7, a flow path is defined through the actuator for passage of propellant and medicament from the canister, the flow path including the expansion chamber, fluid passage and the delivery opening, and wherein at least part of the flow path is constructed from a conductive material having at least one of: a thermal conductivity greater than or equal to 1 W/mK; and an electrical resistivity less than or equal to 1×10¹⁰ Ω·m.

In aspect D9 according to any of aspects D1 to D8, the second end of the delivery tube has a profile with a first dimension in a first direction and a second dimension in a second direction, wherein the first dimension is larger than the second dimension.

In aspect D10 according to any of aspects D1 to D9, the pressurised propellant comprises at least 25% CO₂.

In aspect D11 according to any of aspects D1 to D10, the nosepiece is formed to aim the delivery outlet towards at least one of the turbinates region and the nasopharynx.

In aspect D12 according to aspect D11, the fluid passage of the nosepiece has a first flow axis proximate the valve block, and a second flow axis proximate the delivery opening, wherein the first and second flow axes are at an angle to each other.

In independent aspect E1, a device for delivery of a medicament to at least one of the olfactory region, the turbinates region and the nasopharynx of a nasal cavity as described herein comprising: a canister comprising: a container containing a pressurised propellant and the medicament; and a metering valve configured to release a predetermined amount of the pressurised propellant and the medicament; and an actuator comprising: a housing receiving the canister; a valve block receiving the metering valve, the valve block defining an expansion chamber for passage of propellant and medicament expelled from the canister; and a nosepiece for insertion into a user's nostril, the nosepiece comprising a fluid passage and a delivery opening for expulsion of the propellant and medicament into the user's nostril; wherein the propellant comprises at least 25% CO₂.

In aspect E2 according to aspect E1, a flow path is defined through the actuator for passage of propellant and medicament from the canister, the flow path including the expansion chamber, fluid passage and the delivery opening, and wherein the cross-sectional area of the flow path is greater than the lowest cross-sectional area of the metering valve at all points along the path.

In aspect E3 according to aspect E2, a flow path is defined through the actuator for passage of propellant and medicament from the canister, the flow path including the expansion chamber, fluid passage and the delivery opening, and wherein at least part of the flow path is constructed from a conductive material having at least one of: a thermal conductivity greater than or equal to 1 W/mK; and an electrical resistivity less than or equal to 1×10¹⁰ Ω·m.

In aspect E4 according to any of aspects E1 to E3, the delivery opening has a profile with a first dimension in a first direction and a second dimension in a second direction, wherein the first dimension is larger than the second dimension.

In aspect E5 according to any of aspects E1 to E3, comprising a single delivery tube extending from the valve block to the delivery opening.

In aspect E6 according to aspect E5, the single delivery tube extends from a position within the valve block to the delivery opening, and wherein the single delivery tube has an inlet facing the canister, and a curved portion directing the flow towards an outlet at the delivery opening.

In aspect E7 according to any of aspects E1 to E6, the nosepiece is formed to aim the delivery outlet towards at least one of the turbinates region and the nasopharynx.

In aspect E8 according to aspect E7, wherein the fluid passage of the nosepiece has a first flow axis proximate the valve block, and a second flow axis proximate the delivery opening, wherein the first and second flow axes are at an angle to each other.

It will be noted that the present disclosure encompasses these combined improvements as well as any combinations of the variations thereof described herein.

1. A device for delivery of a medicament to at least one of the olfactory region, the turbinates region or the nasopharynx of a nasal cavity, the device comprising:
a canister comprising:
a container containing a pressurised propellant and the medicament; and
a metering valve configured to release a predetermined amount of the pressurised propellant and the medicament; and
an actuator comprising:
a housing receiving the canister;
a valve block receiving the metering valve, the valve block defining an expansion chamber for passage of propellant and medicament expelled from the canister; and
a nosepiece for insertion into a user's nostril, the nosepiece comprising a fluid passage and a delivery opening for expulsion of the propellant and medicament into the user's nostril;
wherein a flow path is defined through the actuator for passage of propellant and medicament from the canister, the flow path including the expansion chamber, the fluid passage and the delivery opening, and wherein the cross-sectional area of the flow path is greater than the lowest cross-sectional area of the metering valve at all points along the flow path.

2. A device according to claim 1, wherein the metering valve comprises a valve stem having a valve stem inlet and a valve stem outlet, wherein the valve stem defines the lowest cross-sectional area of the metering valve.

3. A device according to claim 2, wherein the valve stem inlet has a cross-sectional area of at least 0.4 mm².

4. A device according to claim 2, wherein the valve stem inlet comprises a plurality of openings defined in a sidewall of the valve stem.

5. A device according to claim 4, wherein the plurality of openings define a total cross-sectional area of at least 0.4 mm².

6. A device according to any claim 2, wherein the valve stem inlet comprises a non-circular opening.

7. A device according to claim 1,
wherein at least part of the flow path is constructed from a conductive material having at least one of:
a thermal conductivity greater than or equal to 1 W/mK; or
an electrical resistivity less than or equal to 1×10¹⁰ Ω·m.

8. A device according to claim 1, wherein the pressurised propellant comprises at least 25% CO₂.

9. A device for delivery of a medicament to at least one of the olfactory region, the turbinates region or the nasopharynx of a nasal cavity, the device comprising:
a canister comprising:
a container containing a pressurised propellant and the medicament; and
a metering valve configured to release a predetermined amount of the pressurised propellant and the medicament; and
an actuator comprising:
a housing receiving the canister;
a valve block receiving the metering valve, the valve block defining an expansion chamber for passage of propellant and medicament expelled from the canister; and
a nosepiece for insertion into a user's nostril, the nosepiece comprising a fluid passage and a delivery opening for expulsion of the propellant and medicament into the user's nostril;
wherein a flow path is defined through the actuator for passage of propellant and medicament from the canister, the flow path including the expansion chamber, fluid passage and the delivery opening, and wherein at least part of the flow path is constructed from a conductive material having at least one of:
a thermal conductivity greater than or equal to 1 W/mK; or
an electrical resistivity less than or equal to 1×10¹⁰ Ω·m.

10. A device according to claim 9, wherein the at least part of the flow path constructed from a conductive material includes a portion of the flow path defined in the valve block.

11. A device according to claim 9, wherein the at least part of the flow path constructed from a conductive material includes at least a part of the expansion chamber.

12. A device according to claim 10 or 11, wherein the valve block comprises a valve block body and an insert defining at least part of the flow path defined in the valve block, wherein the insert is constructed from the conductive material, and wherein the valve block body is constructed from a material having at least one of a lower thermal conductivity or higher electrical resistivity than the insert.

13. A device according to claim 12, wherein a single uninterrupted flow path is defined through the nosepiece from the valve block body.

14. A device according to claim 13, wherein the single uninterrupted flow path is defined through a single delivery tube extending from the valve block body, through the nosepiece to the delivery opening.

15. A device according to claim 9, wherein the conductive material comprises a metal.

16. A device according to claim 15, wherein the conductive material comprises aluminium.

17. A device for delivery of a medicament to at one of the olfactory region, the turbinates region and the nasopharynx of a nasal cavity, the device comprising:
a canister comprising:
a container containing a pressurised propellant and the medicament; and
a metering valve configured to release a predetermined amount of the pressurised propellant and the medicament; and
an actuator comprising:
a housing receiving the canister;
a valve block receiving the metering valve, the valve block defining an expansion chamber for passage of propellant and medicament expelled from the canister; and
a nosepiece for insertion into a user's nostril, the nosepiece comprising a fluid passage and a delivery opening for expulsion of the propellant and medicament into the user's nostril;
wherein the delivery opening has a profile with a first dimension in a first direction and a second dimension in a second direction, wherein the first dimension is larger than the second dimension.

18. A device according to claim 17, wherein the delivery opening has a curved profile.

19. A device according to claim 17, wherein the delivery opening is elliptical.

20. A device according to claim 17, wherein a flow path is defined through the actuator for passage of propellant and medicament from the canister to the delivery opening, and wherein the cross-sectional area of the flow path transitions from a circular profile to the profile of the delivery opening.
